SEQUENCE LISTING (10 sequences)

SEQ ID NO: 1 - protein, 213 aa, Artificial Sequence
  Wild type sequence with N-terminal SPIRR
SPIRRRIPGFDISGWQPTTDFARAYANGDRFVYIKATEGTTFKSSAFSRQYTGATQNGFIRGAYHFAQPAASSGAAQARYFASNGGGWSKDGITLPGALDIEYNPNGATCYGLSQSAMVNWIEDFVTTYHGITSRWPVIYTTTDWWTQCTGNSNRFANRCPLWIARYASSVGTLPNGWGFYTFWQYNDKYPQGGDSNWFNGDASRLRALANGD

SEQ ID NO: 2 - DNA, 946 bp, Trichophaea saccata
  CDS (1)..(347)
  sig_peptide (1)..(51)
  mat_peptide (52)..(943)
  CDS (401)..(615)
  CDS (668)..(772)
  CDS (825)..(943)
atgcacgctctcacccttctcaccgcaaccctcttcggtctcgcagcggcctacccagtgaagaccgaccttcactgccgctcctctcccagcacttccgccagcatcgtccgcacctactccagtggaacggaagtccagatccagtgccagaccacgggcacttcggtccaaggatccaatgtctgggacaagacccagcacggttgctacgtcgcagactactacgtcaagaccgggcattctgggattttcaccaccaagtgcggtagcagctcgggtggaggttcctgcaagcctcccccgatcaatgctgctactgtcgcattgatcaaggagtttgaggggtaagtgacagctctgagtgaggtggtatgaggattaagactgacgaggatagattcgttcctaagcccgccccggatcctattggattgccgaccgtgggatacgggcatctttgcaagactaagggctgcaaagaagtgccttacagcttccctctcacccaggagactgccaccaagttgcttcagagcgatatcaagactttcacctcttgcgttagcaactacgtcaaggactctgttaagctcaacgataaccagtacggaggtgagttccagtgtaacagtgaatttattgatgatattctaagtaattttagctctggcgtcttgggctttcaacgtcggctgcggaaacgtccagacttcttcgctgatcaagagattgaacgctggggagaaccctaacactgtcgctgctcagggtaagatatttatcccggatttgctcttgacacatggctgaaaaagttgcagaactccccaagtggaagtacgctggtggaaaggttatgcctggcttggtccgccgccgcaatgctgaggtcgcgctcttcaagaagcccagcagcgttcaggcccaccctcccaagtgctaa

SEQ ID NO: 3 - protein, 262 aa, Trichophaea saccata
MHALTLLTATLFGLAAAYPVKTDLHCRSSPSTSASIVRTYSSGTEVQIQCQTTGTSVQGSNVWDKTQHGCYVADYYVKTGHSGIFTTKCGSSSGGGSCKPPPINAATVALIKEFEGFVPKPAPDPIGLPTVGYGHLCKTKGCKEVPYSFPLTQETATKLLQSDIKTFTSCVSNYVKDSVKLNDNQYGALASWAFNVGCGNVQTSSLIKRLNAGENPNTVAAQELPKWKYAGGKVMPGLVRRRNAEVALFKKPSSVQAHPPKC

SEQ ID NO: 4 - protein, 245 aa, Trichophaea saccata
  mat_peptide (1)..(245)
YPVKTDLHCRSSPSTSASIVRTYSSGTEVQIQCQTTGTSVQGSNVWDKTQHGCYVADYYVKTGHSGIFTTKCGSSSGGGSCKPPPINAATVALIKEFEGFVPKPAPDPIGLPTVGYGHLCKTKGCKEVPYSFPLTQETATKLLQSDIKTFTSCVSNYVKDSVKLNDNQYGALASWAFNVGCGNVQTSSLIKRLNAGENPNTVAAQELPKWKYAGGKVMPGLVRRRNAEVALFKKPSSVQAHPPKC

SEQ ID NO: 5 - protein, 129 aa, Gallus gallus [sequence omitted]

SEQ ID NO: 6 - DNA, 39 bp, Artificial Sequence
  Primer F-80470
acacaactggggatccaccatgcacgctctcacccttct

SEQ ID NO: 7 - DNA, 38 bp, Artificial Sequence
  Primer R-80470.
ctagatctcgagaagcttttagcacttgggagggtggg

SEQ ID NO: 8 - DNA, 20 bp, Artificial Sequence
  Primer 8643.
gcaagggatgccatgcttgg

SEQ ID NO: 9 - DNA, 19 bp, Artificial Sequence
  Primer 8654.
catataaccaattgccctc

SEQ ID NO: 10 - protein, 213 aa, Acremonium alcalophilum; identical to SEQ ID NO: 1

(19) United States
(12) Patent Application Publication
(10) Pub. No.: US 2024/0156921 A1
(43) Pub. Date: May 16, 2024

(54) ANIMAL FEED COMPOSITION AND USE THEREOF

(71) Applicants: DSM IP Assets B.V., Heerlen (NL); Novozymes A/S, Bagsvaerd (DK)

(72) Inventors: Wolfgang SCHLIFFKA, Kaiseraugst (CH); Rual LOPEZ ULIBARRI, Kaiseraugst (CH); Estefania PEREZ CALVO, Kaiseraugst (CH)

(21) Appl. No.: 18/550,313

(22) PCT Filed: Mar. 14, 2022
(86) PCT No.: PCT/EP2022/056436

§ 371 Date: Sep. 13, 2023

(30) Foreign Application Priority Data
Mar. 16, 2021 (EP) 21162898.7

(51) Int. Cl. A61K 38/47; A23K 20/189

(52) U.S. Cl. CPC A61K 38/47; A23K 20/189; C12Y 302/01017

(57) ABSTRACT

The present invention relates to a method for improving health status and/or performance of female animals during gestation and/or lactation and young animals thereof comprising administering to the female animals one or more microbial muramidases. The present invention also provides a feed composition for the above method and use thereof.

### BACKGROUND OF THE INVENTION

### Field of the Invention

The present invention relates to a method for improving health status and performance of animals during gestation and/or lactation.

### Description of the Related Art

Muramidase, also named as lysozyme, is an O-glycosyl hydrolase produced as a defensive mechanism against bacteria by many organisms. The enzyme causes the hydrolysis of bacterial cell walls by cleaving the glycosidic bonds of peptidoglycan, an important structural molecule in bacteria. After having their cell walls weakened by muramidase action, bacterial cells lyse as a result of umbalanced osmotic pressure.

Muramidase naturally occurs in many organisms such as viruses, plants, insects, birds, reptiles and mammals. Muramidase has been classified into five different glycoside hydrolase (GH) families (CAZy, www.cazy.org): hen egg-white muramidase (GH22), goose egg-white muramidase (GH23), bacteriophage T4 muramidase (GH24), Sphingomonas flagellar protein (GH73) and Chalaropsis muramidases (GH25). Muramidases from the families GH23 and GH24 are primarily known from bacteriophages and have only recently been identified in fungi. The muramidase family GH25 has been found to be structurally unrelated to the other muramidase families.

Muramidase has traditionally been extracted from hen egg white due to its natural abundance and until very recently hen egg white muramidase was the only muramidase investigated for use in animal feed. Muramidase extracted from hen egg white is the primary product available on the commercial market, but does not cleave N,6-O-diacetylmuramic acid in e.g. Staphylococcus aureus cell walls and is thus unable to lyse this important human pathogen among others (Masschalck B, Deckers D, Michiels C W (2002), “Lytic and nonlytic mechanism of inactivation of gram-positive bacteria by muramidase under atmospheric and high hydrostatic pressure”, J Food Prot. 65(12):1916-23).

WO2000/21381 discloses a composition comprising at least two antimicrobial enzymes and a polyunsaturated fatty acid, wherein one of the antimicrobial enzymes was a GH22 muramidase from chicken egg white. GB2379166 discloses a composition comprising a compound that disrupts the peptidoglycan layer of bacteria and a compound that disrupts the phospholipid layer of bacteria, wherein the peptidoglycan disrupting compound was a GH22 muramidase from chicken egg white.

WO2004/026334 discloses an antimicrobial composition for suppressing the growth of enteric pathogens in the gut of livestock comprising (a) a cell wall lysing substance or its salt, (b) a antimicrobial substance, (c) a sequestering agent and (d) a lantibiotic, wherein the cell wall lysing substance or its salt is a GH22 muramidase from hen egg white.

Surprisingly, the inventors of the present invention discovered that muramidases supplemented into a lactation diet provide beneficial effects on health status and performance of animals during gestation and/or lactation.

### SUMMARY OF THE INVENTION

Accordingly, the present invention provides a method for improving health status and/or performance of female animals during gestation and/or lactation and young animals thereof comprising administering to the female animals one or more microbial muramidases.

### OVERVIEW OF SEQUENCE LISTING

SEQ ID NO: 1 is the mature amino acid sequence of a wild type GH25 muramidase from Acremonium alcalophilum with N-terminal SPIRR as described in WO 2013/076253.
SEQ ID NO: 2 is the gene sequence of the GH24 muramidase as isolated from Trichophaea saccata.
SEQ ID NO: 3 is the amino acid sequence as deduced from SEQ ID NO: 2.
SEQ ID NO: 4 is the mature amino acid sequence of a wild type GH24 muramidase from Trichophaea saccata.
SEQ ID NO: 5 is the mature amino acid sequence of a wild type GH22 muramidase from Gallus gallus (hen egg white muramidase).
SEQ ID NO: 6 is primer F-80470.
SEQ ID NO: 7 is primer R-80470.
SEQ ID NO: 8 is primer 8643.
SEQ ID NO: 9 is primer 8654.
SEQ ID NO: 10 is the mature amino acid sequence of a wild type GH25 muramidase from Acremonium alcalophilum as described in WO 2013/076253.

### Definitions

Microbial muramidase: The term “microbial muramidase” means a polypeptide having muramidase activity which is obtained or obtainable from a microbial source. Examples of microbial sources are fungi; i.e. the muramidase is obtained or obtainable from the kingdom Fungi, wherein the term kingdom is the taxonomic rank. In particular, the the microbial muramidase is obtained or obtainable from the phylum Ascomycota, such as the sub-phylum Pezizomycotina, wherein the terms phylum and sub-phylum is the taxonomic ranks.

If the taxonomic rank of a polypeptide is not known, it can easily be determined by a person skilled in the art by performing a BLASTP search of the polypeptide (using e.g. the National Center for Biotechnology Information (NCIB) website http://www.ncbi.nlm.nih.gov/) and comparing it to the closest homologues. An unknown polypeptide which is a fragment of a known polypeptide is considered to be of the same taxonomic species. An unknown natural polypeptide or artificial variant which comprises a substitution, deletion and/or insertion in up to 10 positions is considered to be from the same taxonomic species as the known polypeptide.

Muramidase activity: The term “muramidase activity” means the enzymatic hydrolysis of the 1,4-beta-linkages between N-acetylmuramic acid and N-acetyl-D-glucosamine residues in a peptidoglycan or between N-acetyl-D-glucosamine residues in chitodextrins, resulting in bacteriolysis due to osmotic pressure. Muramidase belongs to the enzyme class EC 3.2.1.17. Muramidase activity is typically measured by turbidimetric determination. The method is based on the changes in turbidity of a suspension of Micrococcus luteus ATCC 4698 induced by the lytic action of muramidase. In appropriate experimental conditions these changes are proportional to the amount of muramidase in the medium (c.f. INS 1105 of the Combined Compendium of Food Additive Specifications of the Food and Agriculture Organisation of the UN (www.fao.org)). For the purpose of the present invention, muramidase activity is determined according to the turbidity assay described in example 5 (“Determination of Muramidase Activity”). In one aspect, the polypeptides of the present invention have at least 20%, e.g., at least 40%, at least 50%, at least 60%, at least 70%, at least 80%, at least 90%, at least 95%, or at least 100% of the muramidase activity of SEQ ID NO: 1. In one aspect, the polypeptides of the present invention have at least 20%, e.g., at least 40%, at least 50%, at least 60%, at least 70%, at least 80%, at least 90%, at least 95%, or at least 100% of the muramidase activity of SEQ ID NO: 4. In one aspect, the polypeptides of the present invention have at least 20%, e.g., at least 40%, at least 50%, at least 60%, at least 70%, at least 80%, at least 90%, at least 95%, or at least 100% of the muramidase activity of SEQ ID NO: 10.

Fragment: The term “fragment” means a polypeptide or a catalytic domain having one or more (e.g., several) amino acids absent from the amino and/or carboxyl terminus of a mature polypeptide or domain; wherein the fragment has muramidase activity. In one aspect, a fragment comprises at least 170 amino acids, such as at least 175 amino acids, at least 177 amino acids, at least 180 amino acids, at least 185 amino acids, at least 190 amino acids, at least 195 amino acids or at least 200 amino acids of SEQ ID NO: 1 and has muramidase activity.

In another aspect, a fragment comprises at least 210 amino acids, such as at least 215 amino acids, at least 220 amino acids, at least 225 amino acids, at least 230 amino acids, at least 235 amino acids or at least 240 amino acids of SEQ ID NO: 4 and has muramidase activity.

In one aspect, a fragment comprises at least 170 amino acids, such as at least 175 amino acids, at least 177 amino acids, at least 180 amino acids, at least 185 amino acids, at least 190 amino acids, at least 195 amino acids or at least 200 amino acids of SEQ ID NO: 10 and has muramidase activity.

Isolated: The term “isolated” means a substance in a form that environment does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., multiple copies of a gene encoding the substance; use of a stronger promoter than the promoter naturally associated with the gene encoding the substance). An isolated substance may be present in a fermentation broth sample.

Mature polypeptide: The term “mature polypeptide” means a polypeptide in its final form following translation and any post-translational modifications, such as N-terminal processing, C-terminal truncation, glycosylation, phosphorylation, etc.

Sequence identity: The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the—nobrief option) is used as the percent identity and is calculated as follows:

(Identical Residues×100)/(Length of Alignment−Total Number of Gaps in Alignment)

Variant: The term “variant” means a polypeptide having muramidase activity comprising an alteration, i.e., a substitution, insertion, and/or deletion, of one or more (several) amino acid residues at one or more (e.g., several) positions. A substitution means replacement of the amino acid occupying a position with a different amino acid; a deletion means removal of the amino acid occupying a position; and an insertion means adding 1, 2, or 3 amino acids adjacent to and immediately following the amino acid occupying the position.

In one aspect, a muramidase variant according to the invention may comprise from 1 to 5; from 1 to 10; from 1 to 15; from 1 to 20; from 1 to 25; from 1 to 30; from 1 to 35; from 1 to 40; from 1 to 45; or from 1-50, i.e. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 alterations and have at least 20%, e.g., at least 40%, at least 50%, at least 60%, at least 70%, at least 80%, at least 90%, at least 95%, or at least 100% of the muramidase activity of the parent muramidase, such as SEQ ID NO: 1, SEQ ID NO: 4 or SEQ ID NO: 10.

Animal feed: The term “animal feed” refers to any compound, preparation, or mixture suitable for, or intended for intake by an animal. Animal feed for a monogastric animal typically comprises concentrates as well as vitamins, minerals, enzymes, direct fed microbial, amino acids and/or other feed ingredients (such as in a premix) whereas animal feed for ruminants generally comprises forage (including roughage and silage) and may further comprise concentrates as well as vitamins, minerals, enzymes direct fed microbial, amino acid and/or other feed ingredients (such as in a premix).

Concentrates: The term “concentrates” means feed with high protein and energy concentrations, such as fish meal, molasses, oligosaccharides, sorghum, seeds and grains (either whole or prepared by crushing, milling, etc. from e.g. corn, oats, rye, barley, wheat), oilseed press cake (e.g. from cottonseed, safflower, sunflower, soybean (such as soybean meal), rapeseed/canola, peanut or groundnut), palm kernel cake, yeast derived material and distillers grains (such as wet distillers grains (WDS) and dried distillers grains with solubles (DDGS)).

Forage: The term “forage” as defined herein also includes roughage. Forage is fresh plant material such as hay and silage from forage plants, grass and other forage plants, seaweed, sprouted grains and legumes, or any combination thereof. Examples of forage plants are Alfalfa (lucerne), birdsfoot trefoil, brassica (e.g. kale, rapeseed (canola), rutabaga (swede), turnip), clover (e.g. alsike clover, red clover, subterranean clover, white clover), grass (e.g. Bermuda grass, brome, false oat grass, fescue, heath grass, meadow grasses, orchard grass, ryegrass, Timothy-grass), corn (maize), millet, barley, oats, rye, sorghum, soybeans and wheat and vegetables such as beets. Forage further includes crop residues from grain production (such as corn stover; straw from wheat, barley, oat, rye and other grains); residues from vegetables like beet tops; residues from oilseed production like stems and leaves form soy beans, rapeseed and other legumes; and fractions from the refining of grains for animal or human consumption or from fuel production or other industries.

Roughage: The term “roughage” means dry plant material with high levels of fiber, such as fiber, bran, husks from seeds and grains and crop residues (such as stover, copra, straw, chaff, sugar beet waste).

### DETAILED DESCRIPTION OF THE INVENTION

Methods Improving Health Status and/or Performance of Animals

It has been surprisingly found that supplementing an animal feed with a microbial muramidase results in a significant benefit of improving health status and/or performance of a female animal during gestation and/or lactation and yong animals thereof, compared to an animal feed without the microbial muramidase.

Thus, as the first aspect, the present invention relates to a method for improving health status and/or performance of a female animal during gestation and/or lactation and yong animals thereof, comprising administering to the female animal one or more microbial muramidases.

Particularly, the present invention relates to a method for improving health status of a female animal during gestation and/or lactation, which means reducing weight gain loss and/or backfat loss of the female animal, comprising administering to the female animal one or more microbial muramidases.

The present invention also relates to a method for improving performance, including reproductive performance and growth performance, of a female animal, which means increasing litter size of yong animals born by the female animal and increasing milk yield of the female animal, comprising administering to the female one or more microbial muramidases.

The present invention further relates to a method for improving performance of yong animals born by a female animal, which means improving weight gain and/or vadility of the yong animal, comprising administering to the female animal one or more microbial muramidases.

In the present invention, the weight gain loss and/or backfat loss of the female animal, such as sows or gilts, may be reduced by at least 0.1%, 0.2%, 0.3%, 0.5%, 0.6%, 0.8% or 1%.

In the present invention, the litter size of the young animal such as piglets may be increased by at least 0.5%, 1%, 1.5%, 2% or 3%.

In the present invention, the milk yield of the female animal such as sows or gilts may be increased by at least 1%, 1.5%, 2%, 2.5%, 3%, 3.5%, 4%, 4.5% or 5%.

In the present invention, the weight gain of the young animal such as piglets may be increased by 1%, 1.5%, 2%, 2.5%, 3%, 3.5%, 4% or 4.5%.

In the present invention, the vadility of the young animal such as piglets may be increased by at least 0.5%, 1%, 1.5% or 2%.

In the present invention, the microbial muramidase may be dosed at a level of 100 to 1000 mg enzyme protein per kg animal feed, such as 200 to 900 mg, 300 to 800 mg, 400 to 700 mg, 500 to 600 mg enzyme protein per kg animal feed, or any combination of these intervals.

In the present invention, the animal may be selected from the group consisting of swine such as sows and gilts; poultry such as turkey, duck, quail, guinea fowl, goose, pigeon, and chicken such as hens and pullets; cattle such as cows; cats; dogs; rabbits; horses; camels and sheep. Preferably, the animal is a selected from the group consisting of swine, chicken, cats, dogs and sheep. More preferably, the animal is a selected from the group consisting of sows, gilts, cows, dogs and sheep. The most preferably, the animal is sow or gilt.

In the present invention, the microbial muramidase may be fed to the female animal during gestation and/or during lactation. In one embodiment, the microbial muramidase is fed to the female animal during gestation. In another embodiment, the microbial muramidase is fed to the female animal during lactation. Preferably, the microbial muramidase is fed to the female animal during gestation and lactation. More preferably, the microbial muramidase is fed to sows or gilts during gestation and lactation. The most preferably, the microbial muramidase is fed to sows or gilts from day 7 before farrowing to day 26 post-farrowing.

In the present invention, the microbial muramidase may be of fungal origin. Preferably, the microbial muramidase is obtained or obtainable from the phylum Ascomycota, such as the sub-phylum Pezizomycotina. Preferably, the microbial muramidase comprises one or more domains selected from the list consisting of GH24 and GH25.

In the present invention, the microbial muramidase may have at least 50%, e.g., at least 60%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 1, 4 or 10.

In the present invention, the microbial muramidase may comprise or consist of the amino acid sequence of SEQ ID NO: 1 or an allelic variant thereof; or is a fragment thereof having muramidase activity, wherein the fragment comprises at least 170 amino acids, such as at least 175 amino acids, at least 177 amino acids, at least 180 amino acids, at least 185 amino acids, at least 190 amino acids, at least 195 amino acids or at least 200 amino acids. Preferably, the microbial muramidase comprises or consists of the amino acid sequence of SEQ ID NO: 1 or an allelic variant thereof and a N-terminal and/or C-terminal His-tag and/or HQ-tag. More preferably, the polypeptide comprises or consists of amino acids 1 to 213 of SEQ ID NO: 1.

Alternatively, the microbial muramidase may comprise or consist of the amino acid sequence of SEQ ID NO: 4 or an allelic variant thereof; or is a fragment thereof having muramidase activity, wherein the fragment comprises at least 210 amino acids, such as at least 215 amino acids, at least 220 amino acids, at least 225 amino acids, at least 230 amino acids, at least 235 amino acids or at least 240 amino acids. Preferably, the microbial muramidase comprises or consists of the amino acid sequence of SEQ ID NO: 4 or an allelic variant thereof and a N-terminal and/or C-terminal His-tag and/or HQ-tag. More preferably, the polypeptide comprises or consists of amino acids 1 to 245 of SEQ ID NO: 4.

More alternatively, the microbial muramidase may comprise or consist of the amino acid sequence of SEQ ID NO: 10 or an allelic variant thereof; or is a fragment thereof having muramidase activity, wherein the fragment comprises at least 210 amino acids, such as at least 215 amino acids, at least 220 amino acids, at least 225 amino acids, at least 230 amino acids, at least 235 amino acids or at least 240 amino acids. Preferably, the microbial muramidase comprises or consists of the amino acid sequence of SEQ ID NO: 10 or an allelic variant thereof and a N-terminal and/or C-terminal His-tag and/or HQ-tag. More preferably, the polypeptide comprises or consists of amino acids 1 to 208 of SEQ ID NO: 10.

In the present invention, the microbial muramidase may be a variant of SEQ ID NO: 1, 4 or 10 wherein the variant has muramidase activity and comprises one or more substitutions, and/or one or more deletions, and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions. Preferably, the number of positions comprising one or more amino acid substitutions, and/or one or more amino acid deletions, and/or one or more amino acid insertions or any combination thereof in SEQ ID NO: 1, 4 or 10 is between 1 and 45, such as 1-40, 1-35, 1-30, 1-25, 1-20, 1-15, 1-10 or 1-5 positions. More preferably, the number of positions comprising one or more amino acid substitutions, and/or one or more amino acid deletions, and/or one or more amino acid insertions or any combination thereof in SEQ ID NO: 1, 4 or 10 is not more than 10, e.g., 1, 2, 3, 4, 5, 6, 7, 8, 9 or 10. Further preferably, the number of substitutions, deletions, and/or insertions in SEQ ID NO: 1, 4 or 10 is not more than 10, e.g., 1, 2, 3, 4, 5, 6, 7, 8, 9 or 10. Further preferably, the number of substitutions, preferably conservative substitutions, in SEQ ID NO: 1, 4 or 10 is not more than 10, e.g., 1, 2, 3, 4, 5, 6, 7, 8, 9 or 10. Further preferably, the number of conservative substitutions in SEQ ID NO: 1, 4 or 10 is not more than 10, e.g., 1, 2, 3, 4, 5, 6, 7, 8, 9 or 10.

Any person skilled in the art can understand, the polypeptide of the microbial muramidase may have amino acid changes. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding domain.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for muramidase activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related polypeptide.

The crystal structure of the Acremonium alcalophilum CBS114.92 muramidase was solved at a resolution of 1.3 Å as disclosed in WO 2013/076253. These atomic coordinates can be used to generate a three-dimensional model depicting the structure of the Acremonium alcalophilum CBS114.92 muramidase or homologous structures (such as the variants of the present invention). Using the x/ray structure, amino acid residues D95 and E97 (using SEQ ID NO: 1 for numbering) were identified as catalytic residues.

In one embodiment, the present invention relates to a method for reducing weight gain loss and/or backfat loss of sows or gilts during gestation and/or lactation, comprising administering to the sows or gilts one or more microbial muramidases, wherein:
(a) the microbial muramidase is a GH24 or GH25 muramidase obtained or obtainable from the phylum Ascomycota, and is dosed at a level of 300 to 500 mg enzyme protein per kg animal feed; and
(b) the microbial muramidase is fed on a daily basis during gestation and lactation of the sows or gilts; and
(c) optionally the weight gain loss and/or backfat loss of the sows or gilts is reduced by at least 1%.

In a second embodiment, the present invention relates to a method for increasing litter size of piglets born by sows or gilts, and/or increasing milk yield of sows or gilts, comprising administering to the sows or gilts one or more microbial muramidases, wherein:

(a) the microbial muramidase is a GH24 or GH25 muramidase obtained or obtainable from the phylum Ascomycota, and is dosed at a level of 300 to 500 mg enzyme protein per kg animal feed; and

(b) the microbial muramidase is fed on a daily basis during gestation and lactation of the sows or gilts; and

(c) optionally, the litter size of the piglets is increased by at least 3%, or the increasing milk yield of sows or gilts is increased by at least 4%.

In a third embodiment, the present invention relates to a method for improving weight gain and/or vadility of piglets, comprising administering to sows or gilts one or more microbial muramidases, wherein:

(a) the microbial muramidase is a GH24 or GH25 muramidase obtained or obtainable from the phylum Ascomycota, and is dosed at a level of 300 to 500 mg enzyme protein per kg animal feed; and

(b) the microbial muramidase is fed on a daily basis during lactation of the sows or gilts; and

(c) optionally, weight gain and/or vadility of piglets is increased by at least 2%.

### Feed Composition, Feed Additive and Animal Feed

The microbial muramidase of the present invention may be formulated as a feed composition for improving health status and/or performance of a female animal during gestation and/or lactation and yong animals thereof, which is also the present invention intents to cover.

Accordingly, as the second asepce, the present invention provides a feed composition comprising one or more microbial muramidases for improving health status and/or performance of a female animal during gestation and/or lactation and yong animals thereof.

Particularly, the present invention provides a feed composition comprising one or more microbial muramidases for reducing weight gain loss and/or backfat loss of a female animal during gestation and/or lactation.

The present invention also provides a feed composition comprising one or more microbial muramidases for increasing litter size of yong animals born by a female animal and/or increasing milk yield of a female animal.

The present invention further provides a feed composition comprising one or more microbial muramidases for improving weight gain and/or vadility of a yong animal during lactation.

In the present invention, the feed composition, and/or the components such as the muramidase which the composition contains may be formulated as a liquid formulation or a solid formulation.

Accordingly, the feed composition according to the present invention may also comprise one or more formulating agents.

The formulating agents may be selected from the group consisting of polyol such as glycerol, sorbitol, ethylene glycol, diethylene glycol, triethylene glycol, 1,2-propylene glycol, 1,3-propylene glycol, dipropylene glycol and polyethylene glycol (PEG); a salt such as organic or inorganic zinc, sodium, potassium, calcium or magnesium salts (for example, magnesium sulfate, calcium acetate, calcium benzoate, calcium carbonate, calcium chloride, calcium citrate, calcium sorbate, calcium sulfate, potassium acetate, potassium benzoate, potassium carbonate, potassium chloride, potassium citrate, potassium sorbate, potassium sulfate, sodium acetate, sodium benzoate, sodium carbonate, sodium chloride, sodium citrate, sodium sulfate, zinc acetate, zinc benzoate, zinc carbonate, zinc chloride, zinc citrate, zinc sorbate and zinc sulfate); and starch or a sugar or sugar derivative such as sucrose, dextrin, glucose, lactose and sorbitol; small organic molecules, flour, cellulose and minerals and clay minerals (also known as hydrous aluminium phyllosilicates such as kaolinite or kaolin).

The feed composition according to the present invention may also comprise one or more emulsifying agents. The emulsifying agents may be selected advantageously from the group consisting of polyglycerol esters of fatty acids such as esterified ricinoleic acid or propylene glycol esters of fatty acids, saccharo-esters or saccharo-glycerides, polyethylene glycol, lecithins, etc.

In the feed composition of the present invention, the microbial muramidase may be provided at a level of 100 to 1000 mg enzyme protein per kg animal feed, such as 200 to 900 mg, 300 to 800 mg, 400 to 700 mg, 500 to 600 mg enzyme protein per kg animal feed, or any combination of these intervals.

As anticipated by any person skilled in the art, the feed composition according to the present invention may be formulated as an animal feed additive. Accordingly, the feed composition of the present invention may also include micro-ingredients.

The micro-ingredients include but are not limited to aroma compounds; antimicrobial peptides; polyunsaturated fatty acids (PUFAs); reactive oxygen generating species; at least one enzyme, and fat- and water-soluble vitamins, as well as minerals.

Examples of antimicrobial peptides (AMP'S) are CAP18, leucocin A, protegrin-1, thanatin, defensin, lactoferrin, lactoferricin, and ovispirin such as novispirin (Robert Lehrer, 2000), plectasins, and statins.

Examples of polyunsaturated fatty acids are C18-, C20- and C22-polyunsaturated fatty acids, such as arachidonic acid, docosohexaenoic acid, eicosapentaenoic acid and gamma-linoleic acid.

Examples of reactive oxygen generating species are chemicals such as perborate, persulphate, or percarbonate; and enzymes such as an oxidase, an oxygenase or a syntethase.

Examples of enzyme are phytase (EC 3.1.3.8 or 3.1.3.26), galactanase (EC 3.2.1.89), alpha-galactosidase (EC 3.2.1.22), phospholipase A1 (EC 3.1.1.32), phospholipase A2 (EC 3.1.1.4), lysophospholipase (EC 3.1.1.5), phospholipase C (EC 3.1.4.3), and/or phospholipase D (EC 3.1.4.4).

Examples of fat-soluble vitamins include but are not limited to vitamin A, vitamin D3, and vitamin K, e.g. vitamin K3.

Examples of water-soluble vitamins include but are not limited to vitamin B12, biotin and choline, vitamin B1, vitamin B2, vitamin B6, niacin, folic acid and panthothenate, e.g. Ca-D-panthothenate.

Examples of minerals include but are not limited to calcium, phosphorus, sodium, potassium, magnesium, chlorine, iodine, iron, manganese, copper, molybdenum, cobalt and zinc. Common mineral supplements in feed are: limestone, Bone meal, oyster shell, sodium chloride, dicalcium phosphate, manganese sulphate, potassium iodide, and superphosphate. Sources of minerals include meat scraps, fish meal, milk products, ground limestone (calcium), ground oyster shells (calcium), dicalcium phosphate (calcium, phosphorus), defluorinated rock phosphate (phosphorus, calcium), steamed bone meal (phosphorus, calcium), salt (sodium, chlorine, iodine), manganese sulfate (manganese), manganese oxide (manganese), zinc carbonate (zinc), zinc oxide (zinc).

As also anticipated by any person skilled in the art, the feed composition according to the present invention may further be formulated as an animal feed. Accordingly, the feed composition of the present invention may further include any number of components typical for an animal feed, such as proteins, carbohydrates as defined above, fats and additional additives.

Examples of suitable types of proteins that can be included in the feed include, but are not limited to, meat scraps (lysine), fish meal (lysine, methionine), poultry by-product meal (tryptophan, lysine), blood meal, liver and glandular meal, feather meal (hydrolyzed), animal tankage, milk products, cottonseed meal, peanut meal, soybean meal, sesame meal, sunflower seed meal.

Most feed ingredients (maize, barley, safflower, milo, wheat, rice, bran, etc.) contain approximately 2-5% fat and linoleic acid. Sources of fats include animal tallow (beef), lard, corn oil, and other vegetable oils.

Additional additives include but are not limited to minerals as defined above; antioxidants like BHT (Butylated hydroxytoluene), santoquin, ethoxyquin, butylated hydroxyanisode and diphenyl paraphenyl diamine; pellet binders such as sodium bentonite (clay), liquid or solid by-products of the wood pulp industry, molasses, and guarmeal; coloring agents such as xanthophylls, synthetic carotinoid, and canthaxanthin; probiotics such as strains of lactobacillus and streptococcus; and/or antibiotics such as penicillin, streptomycin, tetracyclines, and aureomycin.

### Use of Muramidase

As the third aspect, the invention relates to use of one or more muramidase in a feed composition, a feed additive or an animal feed for improving health status and/or performance of a female animal during gestation and/or lactation and yong animals thereof.

Particularly, the present invention relates to use of one or more muramidase in a feed composition, a feed additive or an animal feed for reducing weight gain loss and/or backfat loss of a female animal during gestation and/or lactation.

The present invention relates to use of one or more muramidase in a feed composition, a feed additive or an animal feed for increasing litter size of yong animals born by a female animal and increasing milk yield of a female animal.

The present invention relates to use of one or more muramidase in a feed composition, a feed additive or an animal feed for improving weight gain and/or vadility of a yong animal at least during lactation.

In the present invention, the weight gain loss and/or backfat loss of the female animal, such as sows or gilts, may be reduced by at least 0.1%, 0.2%, 0.3%, 0.5%, 0.6%, 0.8% or 1%.

In the present invention, the litter size of the young animal such as piglets may be increased by at least 0.5%, 1%, 1.5%, 2% or 3%.

In the present invention, the milk yield of the female animal such as sows or gilts may be increased by at least 1%, 1.5%, 2%, 2.5%, 3%, 3.5%, 4%, 4.5% or 5%.

In the present invention, the weight gain of the young animal such as piglets may be increased by 1%, 1.5%, 2%, 2.5%, 3%, 3.5%, 4% or 4.5%.

In the present invention, the vadility of the young animal such as piglets may be increased by at least 0.5%, 1%, 1.5% or 2%.

In the present invention, the microbial muramidase may be dosed at a level of 100 to 1000 mg enzyme protein per kg animal feed, such as 200 to 900 mg, 300 to 800 mg, 400 to 700 mg, 500 to 600 mg enzyme protein per kg animal feed, or any combination of these intervals.

In the present invention, the animal may be selected from the group consisting of swine such as sows and gilts; poultry such as turkey, duck, quail, guinea fowl, goose, pigeon, and chicken such as hens and pullets; cattle such as cows; cats; dogs; rabbits; horses; camels and sheep. Preferably, the animal is a selected from the group consisting of swine, chicken, cats, dogs and sheep. More preferably, the animal is a selected from the group consisting of sows, gilts, cows, dogs and sheep. The most preferably, the animal is sow or gilt.

In the present invention, the microbial muramidase may be fed to the female animal during gestation and/or during lactation. In one embodiment, the microbial muramidase is fed to the female animal during gestation. In another embodiment, the microbial muramidase is fed to the female animal during lactation. Preferably, the microbial muramidase is fed to the female animal during gestation and lactation. More preferably, the microbial muramidase is fed to sows or gilts during gestation and lactation. The most preferably, the microbial muramidase is fed to sows or gilts from day 7 before farrowing to day 26 post-farrowing.

In the present invention, the microbial muramidase may be of fungal origin. Preferably, the microbial muramidase is obtained or obtainable from the phylum Ascomycota, such as the sub-phylum Pezizomycotina. Preferably, the microbial muramidase comprises one or more domains selected from the list consisting of GH24 and GH25.

In the present invention, the feed composition, the feed additive and the animal feed are defined as above.

### EXAMPLES

### Strains

Trichophaea saccata CBS804.70 was purchased from the Centraalbureau voor Schimmelcultures (Utrecht, the Netherlands). According to Central Bureau vor Schnimmelkulture, Trichophaea saccata CBS804.70 was isolated from coal spoil tip soil from Staffordshire, England in May 1968.

According to Central Bureau vor Schnimmelkulture, Acremonium alcalophilum CBS 114.92 was isolated by A. Yoneda in 1984 from the sludge of pig faeces compost near Tsukui Lake, Japan.

### Media and Solutions

YP+2% glucose medium was composed of 1% yeast extract, 2% peptone and 2% glucose.

YP+2% maltodextrin medium was composed of 1% yeast extract, 2% peptone and 2% maltodextrin.

PDA agar plates were composed of potato infusion (potato infusion was made by boiling 300 g of sliced (washed but unpeeled) potatoes in water for 30 minutes and then decanting or straining the broth through cheesecloth). Distilled water was then added until the total volume of the suspension was one liter, followed by 20 g of dextrose and 20 g of agar powder. The medium was sterilized by autoclaving at 15 psi for 15 minutes (Bacteriological Analytical Manual, 8th Edition, Revision A, 1998).

LB plates were composed of 10 g of Bacto-Tryptone, 5 g of yeast extract, 10 g of sodium chloride, 15 g of Bacto-agar, and deionized water to 1 liter.

LB medium was composed of 10 g of Bacto-Tryptone, 5 g of yeast extract, 10 g of sodium chloride, and deionized water to 1 liter.

COVE sucrose plates were composed of 342 g of sucrose, 20 g of agar powder, 20 ml of COVE salts solution, and deionized water to 1 liter. The medium was sterilized by autoclaving at 15 psi for 15 minutes (Bacteriological Analytical Manual, 8th Edition, Revision A, 1998). The medium was cooled to 60° C. and 10 mM acetamide, 15 mM CsCl, TRITON® X-100 (50 μl/500 ml) were added.

COVE salts solution was composed of 26 g of MgSO₄·7H₂O, 26 g of KCL, 26 g of KH₂PO4, 50 ml of COVE trace metals solution, and deionized water to 1 liter.

COVE trace metals solution was composed of 0.04 g of Na₂B₄O₇·10H₂O, 0.4 g of CuSO₄·5H₂O, 1.2 g of FeSO₄·7H₂O, 0.7 g of MnSO₄·H₂O, 0.8 g of Na₂MoO₄·2H₂O, 10 g of ZnSO₄·7H₂O, and deionized water to 1 liter.

### Example 1: Cloning, Expression and Purification of the GH25 Muramidase from Acremonium alcalophilum CBS 114.92

The GH25 muramidase from Acremonium alcalophilum CBS 114.92 (SEQ ID NO: 1) was cloned and expressed as described in example 8 and purified as described in example 5 of WO 2013/076253. Alternatively, SEQ ID NO: 10 can be cloned and expressed as described in example 2 of WO 2013/076253.

### Example 2: Expression of the GH24 Muramidase from Trichophaea saccata

The fungal strain was cultivated in 100 ml of YP+2% glucose medium in 1000 ml Erlenmeyer shake flasks for 5 days at 20° C. Mycelia were harvested from the flasks by filtration of the medium through a Buchner vacuum funnel lined with MIRACLOTH® (EMD Millipore, Billerica, MA, USA). Mycelia were frozen in liquid nitrogen and stored at −80° C. until further use. Genomic DNA was isolated using a DNEASY® Plant Maxi Kit (QIAGEN GMBH, Hilden Germany) according to the manufacturer's instructions.

Genomic sequence information was generated by Illumina MySeq (Illumina Inc., San Diego, CA). 5 μgs of the isolated Trichophaea saccata genomic DNA was used for library preparation and analysis according to the manufacturer's instructions. A 100 bp, paired end strategy was employed with a library insert size of 200-500 bp. One half of a HiSeq run was used for the total of 95,744,298, 100 bp raw reads obtained. The reads were subsequently fractionated to 25% followed by trimming (extracting longest sub-sequences having Phred-scores of 10 or more). These reads were assembled using Idba version 0.19. Contigs shorter than 400 bp were discarded, resulting in 8,954,791,030 bp with an N-50 of 10,035. Genes were called using GeneMark.hmm ES version 2.3c and identification of the catalytic domain was made using “Phage muramidase PF00959” Hidden Markov Model provided by Pfam. The polypeptide coding sequence for the entire coding region was cloned from Trichophaea saccata CBS804.70 genomic DNA by PCR using the primers F-80470 and R-80470 (SEQ ID NO: 6 and SEQ ID NO: 7 respectively) as described below.

(SEQ ID NO: 6)
5′-ACACAACTGGGGATCCACCATGCACGCTCTCACCCTTCT-3′
(SEQ ID NO: 7)
5′-CTAGATCTCGAGAAGCTTTTAGCACTTGGGAGGGTGGG-3′

Bold letters represent Trichophaea saccata enzyme coding sequence. Restriction sites are underlined. The sequence to the left of the restriction sites is homologous to the insertion sites of pDau109 (WO 2005/042735).

Extensor HIFI PCR mix, 2× concentration (Thermo Scientific cat no AB-0795) was used for experiment.

The amplification reaction (25 μl) was performed according to the manufacturer's instructions (Thermo Scientific cat no AB-0795) with the following final concentrations:
PCR mix:
0.5 μM Primer F-80470
0.5 μM Primer R-80470
12.5 μl Extensor HIFI PCR mix, 2× conc.
11.0 μl H₂O
10 ng of Trichophaea saccata CBS804.70 genomic DNA.

The PCR reaction was incubated in a DYAD® Dual-Block Thermal Cycler (BioRad, USA) programmed for 1 cycle at 94° C. for 30 seconds; 30 cycles each at 94° C. for 30 seconds, 52° C. for 30 seconds and 68° C. for 60 seconds followed by 1 cycle at 68° C. for 6 minutes. Samples were cooled to 10° C. before removal and further processing.

Three μl of the PCR reaction were analyzed by 1% agarose gel electrophoresis using 40 mM Tris base, 20 mM sodium acetate, 1 mM disodium EDTA (TAE) buffer. A major band of about 946 bp was observed. The remaining PCR reaction was purified directly with an ILLUSTRA™ GFX™ PCR DNA and Gel Band Purification Kit (GE Healthcare, Piscataway, NJ, USA) according to the manufacturer's instructions.

Two μg of plasmid pDau109 was digested with Bam HI and Hind III and the digested plasmid was run on a 1% agarose gel using 50 mM Tris base-50 mM boric acid-1 mM disodium EDTA (TBE) buffer in order to remove the stuffer fragment from the restricted plasmid. The bands were visualized by the addition of SYBR® Safe DNA gel stain (Life Technologies Corporation, Grand Island, NY, USA) and use of a 470 nm wavelength transilluminator. The band corresponding to the restricted plasmid was excised and purified using an ILLUSTRA™ GFX™ PCR DNA and Gel Band Purification Kit. The plasmid was eluted into 10 mM Tris pH 8.0 and its concentration adjusted to 20 ng per μl. An IN-FUSION® PCR Cloning Kit (Clontech Laboratories, Inc., Mountain View, CA, USA) was used to clone the 983 bp PCR fragment into pDau109 digested with Bam HI and Hind III (20 ng). The IN-FUSION® total reaction volume was 10 μl. The IN-FUSION® total reaction volume was 10 μl. The IN-FUSION® reaction was transformed into FUSION-BLUE™ E. coli cells (Clontech Laboratories, Inc., Mountain View, CA, USA) according to the manufacturer's protocol and plated onto LB agar plates supplemented with 50 μg of ampicillin per ml. After incubation overnight at 37° C., transformant colonies were observed growing under selection on the LB plates supplemented with 50 μg of ampicillin per ml.

Several colonies were selected for analysis by colony PCR using the pDau109 vector primers described below. Four colonies were transferred from the LB plates supplemented with 50 μg of ampicillin per ml with a yellow inoculation pin (Nunc A/S, Denmark) to new LB plates supplemented with 50 μg of ampicillin per ml and incubated overnight at 37° C.

Primer 8653:
(SEQ ID NO: 8)
5′-GCAAGGGATGCCATGCTTGG-3′
Primer 8654:
(SEQ ID NO: 9)
5′-CATATAACCAATTGCCCTC-3′

Each of the three colonies were transferred directly into 200 μl PCR tubes composed of 5 μl of 2× Extensor HIFI PCR mix, (Thermo Fisher Scientific, Rockford, IL, USA), 0.5 μl of primer 8653 (10 pm/μl), 0.5 μl of primer 8654 (10 pm/μl), and 4 μl of deionized water. Each colony PCR was incubated in a DYAD® Dual-Block Thermal Cycler programmed for 1 cycle at 94° C. for 60 seconds; 30 cycles each at 95° C. for 30 seconds, 60° C. for 45 seconds, 72° C. for 60 seconds, 68° C. for 10 minutes, and 10° C. for 10 minutes.

Three μl of each completed PCR reaction were submitted to 1% agarose gel electrophoresis using TAE buffer. All four E. coli transformants showed a PCR band of about 980 bp. Plasmid DNA was isolated from each of the four colonies using a QlAprep Spin Miniprep Kit (QIAGEN GMBH, Hilden Germany). The resulting plasmid DNA was sequenced with primers 8653 and 8654 (SEQ ID NO: 8 and 9) using an Applied Biosystems Model 3730 Automated DNA Sequencer using version 3.1 BIG-DYE™ terminator chemistry (Applied Biosystems, Inc., Foster City, CA, USA). One plasmid, designated pKKSC0312-2, was chosen for transforming Aspergillus oryzae MT3568. A. oryzae MT3568 is an amdS (acetamidase) disrupted gene derivative of Aspergillus oryzae JaL355 (WO 2002/40694) in which pyrG auxotrophy was restored by inactivating the A. oryzae amdS gene. Protoplasts of A. oryzae MT3568 were prepared according to the method described in European Patent, EP0238023, pages 14-15.

E. coli 3701 containing pKKSC0312-2 was grown overnight according to the manufacturer's instructions (Genomed) and plasmid DNA of pKKSC0312-2 was isolated using a Plasmid Midi Kit (Genomed JETquick kit, cat.nr. 400250, GENOMED GmbH, Germany) according to the manufacturer's instructions. The purified plasmid DNA was transformed into Aspergillus oryzae MT3568. A. oryzae MT3568 protoplasts were prepared according to the method of Christensen et al., 1988, Bio/Technology 6: 1419-1422. The selection plates consisted of COVE sucrose with +10 mM acetamide+15 mM CsCl+ TRITON® X-100 (50 μl/500 ml). The plates were incubated at 37° C. Briefly, 8 μl of plasmid DNA representing 3 ugs of DNA was added to 100 μl MT3568 protoplasts. 250 μl of 60% PEG solution was added and the tubes were gently mixed and incubate at 37° for 30 minutes. The mix was added to 10 ml of pre melted Cove top agarose (The top agarose melted and then the temperature equilibrated to 40 C in a warm water bath before being added to the protoplast mixture). The combined mixture was then plated on two Cove-sucrose selection petri plates with 10 mM Acetamide. The plates were incubated at 37° C. for 4 days. Single Aspergillus transformed colonies were identified by growth on plates using the selection Acetimide as a carbon source. Each of the four A. oryzae transformants were inoculated into 750 μl of YP medium supplemented with 2% glucose and also 750 μl of 2% maltodextrin and also DAP4C in 96 well deep plates and incubated at 37° C. stationary for 4 days. At the same time the four transformants were restreaked on COVE-2 sucrose agar medium.

Culture broth from the Aspergillus oryzae transformants were then analyzed for production of the GH24 polypeptide by SDS-PAGE using NUPAGE® 10% Bis-Tris SDS gels (Invitrogen, Carlsbad, CA, USA) according to the manufacturer's recommendations. A protein band at approximately 27 kDa was observed for each of the Aspergillus oryzae transformants. One A. oryzae transformant was cultivated in 1000 ml Erlenmeyer shake flasks containing 100 ml of DAP4C medium at 26° C. for 4 days with agitation at 85 rpm.

### Example 3: Purification of the GH24 Muramidase from Trichophaea saccata

The fermentation supernatant with the GH24 muramidase from example 2 was filtered through a Fast PES Bottle top filter with a 0.22 μm cut-off. The resulting solution was diafiltrated with 5 mM Na-acetate, pH 4.5 and concentrated (volume reduced by a factor of 10) on an Ultra Filtration Unit (Sartorius) with a 10 kDa cut-off membrane.

After pretreatment about 275 mL of the muramidase containing solution was purified by chromatography on SP Sepharose (approximately 60 mL) in a XK26 column eluting the bound muramidase with 0 to 100% gradient of buffer A (50 mM Na-acetate pH 4.5) and buffer B (50 mM Na-acetate+1 M NaCl pH 4.5) over 10 column volumes. The fractions from the column were pooled based on the chromatogram (absorption at 280 and 254 nm) and SDS-PAGE analysis.

The molecular weight, as estimated from SDS-PAGE, was approximately 27 kDa and the purity was >90%.

### Example 4: Other Characteristics for the GH24 Muramidase from Trichophaea saccata

Determination of the N-terminal sequence was: YPVKTDL.

The calculated molecular weight from this mature sequence is 26205.5 Da (M+H)+.

The molecular weight determined by intact molecular weight analysis was 26205.3 Da. (M+H)+.

The mature sequence (from EDMAN N-terminal sequencing data, intact molecular weight analysis and proteomic analysis):

(SEQ ID NO: 4)
YPVKTDLHCRSSPSTSASIVRTYSSGTEVQIQCQTTGTSVQGSNVWDKT
QHGCYVADYYVKTGHSGIFTTKCGSSSGGGSCKPPPINAATVALIKEFE
GFVPKPAPDPIGLPTVGYGHLCKTKGCKEVPYSFPLTQETATKLLQSDI
KTFTSCVSNYVKDSVKLNDNQYGALASWAFNVGCGNVQTSSLIKRLNAG
ENPNTVAAQELPKWKYAGGKVMPGLVRRRNAEVALFKKPSSVQAHPPK
C.

### Example 5: Determination of Muramidase Activity

Muramidase activity was determined by measuring the decrease (drop) in absorbance/optical density of a solution of resuspended Micrococcus lysodeikticus ATTC No. 4698 (Sigma-Aldrich M3770) or Exiguobacterium undea (DSM14481) measured in a spectrophotometer at 540 nm.

### Preparation of Micrococcus Lysodeikticus Substrate

Before use the cells were resuspended in citric acid—phosphate buffer pH 6.5 to a concentration of 0.5 mg cells/mL and the optical density (OD) at 540 nm was measured. The cell suspension was then adjusted so that the cell concentration equalled an OD540=1.0. The adjusted cell suspension was then stored cold before use. Resuspended cells were used within 4 hours.

### Preparation of Dried Cells of Exiquobacterium Undae Substrate

A culture of E. undae (DSM14481) was grown in 100 mL LB medium (Fluka 51208, 25 g/L) in a 500 mL shake-flask at 30° C., 250 rpm overnight. The overnight culture was then centrifuged at 20° C. and 5000 g for 10 minutes, and the pellet was then washed twice with sterile milliQ water, and resuspended in MilliQ water. The washed cells were centrifuged for 1 minute at 13000 rpm and as much as possible of the supernatant was decanted. The washed cells were dried in a vacuum centrifuge for 1 hour. The cell pellet was resuspended in citric acid—phosphate buffer pH 4, 5 or 6 so that the optical density (OD) at 540 nm=1.

### Measurement of Muramidase Antimicrobial Activity in the Turbidity Assay

The muramidase sample to be measured was diluted to a concentration of 100-200 mg enzyme protein/L in citric acid—phosphate buffer pH 4, 5 or 6, and kept on ice until use. In a 96 well microtiterplate (Nunc) 200 μL of the substrate was added to each well, and the plate was incubated at 37° C. for 5 minutes in a VERSAmax microplate reader (Molecular Devices). Following incubation, the absorbance of each well was measured at 540 nm (start value). To start the activity measurement, 20 μL of the diluted muramidase sample was added to each substrate (200 μl) and kinetic measurement of absorbance at 540 nm was initiated for minimum 30 minutes up to 24 hours at 37° C. The measured absorbance at 540 nm was monitored for each well and over time a drop in absorbance is seen if the muramidase has muramidase activity. The results are presented in table 1 below.

TABLE 1
Muramidase Activity against Micrococcus lysodeikticus and
Exiguobacterium undea as measured by Optical Density Drop

| | Micrococcus | Exiguobacterium |
|---|---|---|
| Muramidase | lysodeikticus¹ | undae¹ |
| GH22 muramidase from Gallus | +++ (pH 6) | + (pH 6) |
| gallus (SEQ ID NO: 5) | | |
| GH24 muramidase from Trichophaea | ++ (pH 6) | ++ (pH 6) |
| saccata (SEQ ID NO: 4) | | |
| GH25 muramidase from A. | + (pH 4) | + (pH 5) |
| alcalophilum (SEQ ID NO: 1) | | |

¹− Means no effect; + means small effect; ++ means medium effect; +++ means large effect. The pH value in the brackets lists the assay pH based on muramidase-substrate combination.

The data confirms that the GH22 muramidase from Gallus gallus, the GH24 muramidase from Trichophaea saccata and the GH25 muramidase from A. alcalophilum all have muramidase activity.

### Example 6: In Vivo Broiler Trial

### Experimental Objectives

The aim of the study was to test the efficacy of the new feed additive muramidase supplemented into a lactation diet at the recommended dose level (656 mg/kg) of multiparous sows (DanBred) from d 7 before to d 26 post-farrowing (34-d feeding period).

### Experimental Design

The design of the efficacy test was including 2 treatments. The first treatment (T1) was the control group without muramidase addition. In the second treatment (T2) muramidase was added into the lactation feed at the recommended dosage. Details are presented in Table 2.

TABLE 2
Overview of the treatments applied to multiparous
sows from d 7 before to d 26 post-farrowing

| Treatment | | T1 | T2 |
|---|---|---|---|
| Sows | no | 50 | 50 |

Lactation diet (d 7 before
to d 26 post-farrowing)

| | | | |
|---|---|---|---|
| Muramidase ¹⁾ | LSU/kg diet | 0 | 50,000 |
| | mg/kg diet | 0 | 656 |

¹⁾ One LSU unit was defined as the amount of enzyme that increases the fluorescence of a 12.5 mg/mL fluorescein-labelled peptidoglycan suspension by a value that corresponds to the fluorescence of 0.077 mM fluorescein isothiocyanate (FITC), per minute at pH 7.5 and 30° C.

### Animals and Housing

In total, 100 healthy sows of parity 2-8 (5.5±2.3) were allocated to 2 treatments (T1 control, T2 muramidase) such that all treatments were similar in terms of parity and body condition/weight. After mating, sows were housed in gestation pens. Each gestation pen had a front side of 25 separate feeding-resting cubicles with an equal number of automatic feeders providing a daily, individual, pre-weighed feed ration to each pregnant sow. Approximately 9 d before farrowing (d 106 on pregnancy) sows were allocated to farrowing crates for each sow including a creep area for her piglets. All farrowing crates were equipped with nipple drinkers and with separate feeders for sows and piglets. Extra heat was provided to piglets from birth up to weaning, using infrared electric lamps in the creep area. The layout of the experimental farrowing crates was formed to avoid the effect of house and to minimize the risk of cross contamination.

Within 24 hours after farrowing all stillborn, dead, splay-legged, and moribund piglets were removed from the study, leaving only healthy piglets suckling the sow. Cross-fostering equalized litters (13 piglets/litter) and was carried out within 24 h post-farrowing and within the same treatment group.

Throughout the 34-d feeding period farrowing rooms were automatically air ventilated, keeping the temperature between 20 and 23° C. The relative humidity was in the range of 60 to 65%. The house was lit by programmable artificial light. The standard lighting programme was 16 hours of light per day throughout the experimental period. Water was provided ad libitum from nipple drinkers. Piglets were weaned at ca. 26 days of age (±1.5 days) and were moved to the nursery barn. Iron supplementation and castration of male piglets was practiced at day 3 of age.

### Diet Composition

T1 control feed was made first and stored separately from T2 feed. All nutrients were supplied at normal concentrations, not exceeding EU maximum permitted concentrations for trace minerals or vitamins.

Ingredients including vitamin/mineral premixture and calculated analyses of the basal lactation diet is presented in Table 3. Muramidase was added to the basal diet at the expense of Tixosil (>97% silicon dioxide) and salt. All diets in mash form were manufactured in a commercial feed mill.

TABLE 3
Composition and calculated analysis of the lactation feed

| | | |
|---|---|---|
| Treatment group | T1 | T2 |

Ingredients

| | | | |
|---|---|---|---|
| Barley | % | 42.0000 | 42.0000 |
| Wheat | % | 21.5444 | 21.5444 |
| Soybean meal (CP: 49%) | % | 17.2000 | 17.2000 |
| Corn | % | 10.0000 | 10.0000 |
| Soybean oil | % | 3.0000 | 3.0000 |
| Calcium carbonate | % | 1.5500 | 1.5500 |
| Dried sugar beet pulp | % | 1.5000 | 1.5000 |
| Monocalcium phosphate | % | 1.2000 | 1.2000 |
| Vitamin/mineral premix ¹⁾ | % | 1.2000 | 1.2000 |
| L-Lysine HCL | % | 0.4800 | 0.4800 |
| DL-Methionine | % | 0.2000 | 0.2000 |
| L-Threonine | % | 0.0400 | 0.0400 |
| L-Tryptophan | % | 0.0200 | 0.0200 |
| Sodium chloride | % | 0.0392 | |
| Tixosil ²⁾ | % | 0.0264 | |
| Muramidase | % | | 0.0656 |

Calculated analysis

| | | | |
|---|---|---|---|
| ME ³⁾ | MJ/kg | 13.36 | 13.36 |
| Crude protein | g/kg | 170.10 | 170.10 |
| Crude fibre | g/kg | 40.90 | 40.90 |
| Crude fat | g/kg | 49.00 | 49.00 |
| Lysine | g/kg | 11.80 | 11.80 |
| Methionine/Cystine | g/kg | 7.70 | 7.70 |
| Threonine | g/kg | 6.50 | 6.50 |
| Tryptophan | g/kg | 2.20 | 2.20 |
| Calcium | g/kg | 8.80 | 8.80 |
| Dig. Phosphorus | g/kg | 4.00 | 4.00 |
| Sodium | g/kg | 2.12 | 2.12 |

¹⁾ Contents per kg premix: 400,000 I.U. vit. A (acetate); 120,000 I.U. vit. D₃; 8,000 mg vit. E (α-tocopherole acetate); 200 mg vit. K₃ (MSB); 250 mg vit. B₁ (mononitrate); 420 mg vit. B₂ (cryst. riboflavin); 2,500 mg niacin (niacinamide); 400 mg Vit. B₆ (HCl); 2,000 μg vit. B₁₂; 25,000 μg Biotin (commercial, feed grade); 1,000 mg pantothenic acid (Ca d-pantothenate); 100 mg folic acid (cryst. commercial feed < grade); 80,000 mg choline (chloride); 5,000 mg Zn (sulphate); 5,000 mg Fe (carbonate); 6,000 mg Mn (sulphate); 1,000 mg Cu (sulphate-pentahydrate); 20 mg Se (Na-selenite); 45 mg I (Ca-iodate); 130 g Na (NaCl); 55 g Mg (sulphate)
²⁾ Silicon dioxide >97%;
³⁾ Calculated by using the estimation given by DLG 2013.

Lactation feed was given at study start until one day before the expected farrowing in daily amounts of 3.4 kg. At farrowing 1 kg was provided. Afterwards the amount of lactation diet was enhanced continuously until maximum feeding capacity was reached.

The creep feed in mash form (particle size: 0.5 to 3.0 mm) was manufactured and offered ad libitum from d 7 of age up to weaning at d 26 days of age by using automatic feeders accessible only for the piglets. Ingredients including vitamin/mineral premixture and calculated analyses are presented in Table 4.

TABLE 4
Composition and calculated analysis of the creep feed

| Treatment groups | | T1 & T2 |
|---|---|---|
| | % | Ingredients |
| Wheat | % | 35.88 |
| Soybean meal (CP: 49%) | % | 23.00 |
| Schimmer-milk-powder | % | 14.00 |
| Barley | % | 12.00 |
| Corn | % | 10.00 |
| Limestone | % | 1.40 |
| Mineral & vitamin premix¹⁾ | % | 1.20 |
| Monocalcium phosphat | % | 1.00 |
| Soybean oil | % | 1.00 |
| L-Lysine | % | 0.30 |
| DL-Methionine | % | 0.15 |
| L-Tryptophan | % | 0.07 |
| | | Calculated analysis |
| ME ²⁾ | MJ/kg | 13.56 |
| Crude protein | g/kg | 229.00 |
| Lysine | g/kg | 15.20 |
| Methionine/Cystine | g/kg | 9.10 |
| Threonine | g/kg | 8.90 |
| Tryptophan | g/kg | 3.40 |
| Crude fiber | g/kg | 26.00 |
| Crude fat | g/kg | 27.20 |
| Calcium | g/kg | 9.70 |
| Phosphorus | g/kg | 7.10 |
| Digestible phosphorus | g/kg | 4.80 |
| Sodium | g/kg | 1.60 |

¹⁾Contents per kg premix: 400,000 I.U. vit. A (acetate); 120,000 I.U. vit. D₃; 8,000 mg vit. E (α-tocopherole acetate); 200 mg vit. K₃ (MSB); 250 mg vit. B₁ (mononitrate); 420 mg vit. B₂ (cryst. riboflavin); 2,500 mg niacin (niacinamide); 400 mg Vit. B₆ (HCl); 2,000 μg vit. B₁₂; 25,000 μg Biotin (commercial, feed grade); 1,000 mg pantothenic acid (Ca d-pantothenate); 100 mg folic acid (cryst. commercial feed grade); 80,000 mg choline (chloride); 5,000 mg Zn (sulphate); 5,000 mg Fe (carbonate); 6,000 mg Mn (sulphate); 1,000 mg Cu (sulphate-pentahydrate); 20 mg Se (Na-selenite); 45 mg I (Ca-iodate); 130 g Na (NaCl); 55 g Mg (sulphate)
²⁾ Calculated by using the estimation given by DLG 2013.

### Measured Parameters

The following parameters were recorded:

All sows were weighed at 7 d before and at d 26 post-farrowing. Their piglets were weighed litter- or individual-based at birth (litter based), after cross-fostering (d 1 of age) and at weaning (d 26 of age), respectively. The estimation of the dam body weight loss during lactation based on body weight at study start and study end corrected by assuming 25 kg body mass for fetus & adnexa per sow.

Back fat thickness of sows was measured at study start, and at study end by using a ultrasound device about 10 cm left from the backbone midline at the last rib level.

Number of piglets born alive/stillborn, number of piglets/litter after cross-fostering, number & age of piglets at weaning.

Individual feed intake of sows throughout the 34-d feeding period was recorded daily and corrected by possible leftovers. The creep feed intake of suckling piglets was estimated per litter from d 7 of age up to weaning at d 26 of age and presented as daily intake per litter or piglet by dividing the overall feed intake and the number of piglets per litter adjusted to piglet losses.

Health status of sows and their litters, especially occurrence of digestive disorders, abnormal behaviour and clinical signs was recorded daily. Any adverse effect detected during the efficacy trial as well as therapeutic or preventive treatments were recorded as needed. Additionally, the rectal body temperature of sows was measured at one day before and at one day post-farrowing.

### Statistics

Results are presented according to the EFSA Guidance on Statistical reporting (EFSA Journal 2014; 12(12):3908), in particular descriptive statistics following Section 9.2.1; and results of statistical analyses in line with Section 9.2.2). Main analyses results were presented as point estimate and confidence interval.

For all measurements taken at individual- or litter level, the basic statistical technique used was ANOVA with treatment as explanatory variable. Differences were considered significant when P<0.05, whereas P<0.10 were considered a near-significant trend. Analysis were performed with the software package SPSS (IBM SPSS Version 21).

### Results

Sows

The effect of muramidase on body weight, feed intake and back fat in sows during the 34-d feeding period is presented in Table 5.

Due to the selection parity, body weight, and back fat thickness at study start were nearly identical between both treatments. At d 26 post-farrowing sows fed the T2 diet (muramidase) showed an enhanced body weight gain (+1.2%) and a corresponding higher back fat thickness (+1.02%) when compared to the control. Moreover, the feed intake during the 26-d post-arrowing period of sows fed the lactation feed with addition of muramidase seemed to be lower than that recorded in the control group (−3.9%).

TABLE 5
Effects of muramidase on sow characteristics
from d 7 before to d 26 post-farrowing

| Treatment groups | | T1 | T2 | P-value |
|---|---|---|---|---|
| Sows | no | 50 | 50 | |
| Litter number | | 5.6 ± 2.3 | 5.5 ± 2.3 | 0.878 |
| Length of lactation period | days | 26.2 ± 1.4 | 25.8 ± 1.6 | 0.124 |
| Body weight | | | | |
| Study start | kg | 281 ± 57 | 283 ± 64 | 0.899 |
| Study end | kg | 255 ± 55 | 258 ± 64 | 0.845 |
| Body weight loss | kg | −0.83 ± 5.83 | −0.71 ± 4.2 | 0.906 |
| during lactation ¹⁾ | | | | |
| Back fat thickness | | | | |
| Study start | mm | 16.62 ± 1.38 | 16.72 ± 1.43 | 0.723 |
| Study end | mm | 15.70 ± 1.61 | 16.04 ± 1.75 | 0.314 |
| Back fat loss | mm | −0.92 ± 1.38 | −0.68 ± 1.32 | 0.376 |
| Daily feed intake | | | | |
| Pre-farrowing | kg | 3.33 ± 0.22 | 3.26 ± 0.27 | 0.115 |
| Post-farrowing | kg | 5.71 ± 0.82 | 5.49 ± 0.66 | 0.148 |

¹⁾ Corrected by assuming 25 kg for fetus & adnexa.

Litters The effects of muramidase on litter performance during the 26-d suckling period are presented in Table 6.

TABLE 6
Effects of muramidase on litter characteristics

| Treatment groups | | T1 | T2 | P-value |
|---|---|---|---|---|
| Litters | no | 50 | 50 | |
| Born piglets/litter | no | 18.44 ± 4.36 | 19.08 ± 4.04 | 0.590 |
| Stillborn piglets/litter | no | 2.76 ± 2.47 | 3.10 ± 2.79 | 0.521 |
| Live born piglets/litter | no | 15.68 ± 3.59 | 15.98 ± 3.22 | 0.664 |
| Litter weight/live born | kg | 20.52 ± 5.33 | 20.96 ± 4.40 | 0.967 |
| piglets | | | | |
| Fostered piglets/litter | no | 13 ± 0 | 13 ± 0 | 1.000 |
| Litter weight after cross- | kg | 18.53 ± 2.82 | 18.25 ± 2.78 | 0.624 |
| fostering/litter | | | | |
| Piglets weaned/litter | no | 11.7 ± 1.1^{x} | 12.1 ± 0.9^{y} | 0.066 |
| Piglet losses/litter | % | 10.00 ± 8.40^{x} | 7.08 ± 7.26^{y} | 0.066 |
| Litter weight at weaning | kg | 88.05 ± 17.08 | 90.93 ± 13.46 | 0.350 |
| Litter weight gain | kg | 69.52 ± 16.45 | 72.68 ± 12.28 | 0.279 |

^{xy}Different superscripts in same row are trending (x/y 0.05 < P ≤ 0.10).

At birth, no significant differences between treatments in litter weight were noted but the treatment T2 provided higher number of live born piglets. The litter size after cross-fostering amounted to 13 piglets per litter. As a matter of the trend on reduced weaning losses in sows fed diets containing muramidase (10.2 vs. 7.2%, P=0.066), the number of weaned piglets of sows fed the T2 diet (muramidase) was higher when compared to the control (584 vs. 604; corresponding to +3.4%). Consequently, the litter weight gain from cross-fostering up to weaning was on average 4.5 kg greater than that of the control.

### Suckling Piglets

Litter—and individual based performance of suckling piglets are given in Table 7.

TABLE 7
Effects of muramidase on litter- and individual-based performance
of suckling piglets from cross-fostering up to d 26 post-farrowing

| | | | |
|---|---|---|---|
| Treatment groups | T1 | T2 | P-value |

Litter-based

| | | | | |
|---|---|---|---|---|
| Fostered piglets/litter | no | 13 ± 0 | 13 ± 0 | 1.000 |
| Weaned piglets/litter | no | 11.7 ± 1.1^{x} | 12.1 ± 0.9^{y} | 0.066 |
| Body weight | | | | |
| Piglets after cross-fostering | kg | 1.43 ± 0.22 | 1.40 ± 0.21 | 0.624 |
| Piglets at weaning | kg | 7.50 ± 1.18 | 7.52 ± 0.89 | 0.928 |
| Body weight gain | | | | |
| Cumulative | kg | 5.95 ± 1.41 | 5.99 ± 1.18 | 0.840 |
| Daily | g | 232 ± 43 | 238 ± 32 | 0.434 |

Individual-based

| | | | | |
|---|---|---|---|---|
| Fostered piglets | no | 650 | 650 | |
| Weaned piglets | no | 584 | 604 | |
| Body weight | | | | |
| Piglets after cross-fostering | kg | 1.43 ± 0.34 | 1.40 ± 0.21 | 0.356 |
| Piglets at weaning | kg | 7.50 ± 1.98 | 7.50 ± 1.79 | 0.966 |
| Body weight gain | | | | |
| Cumulative | kg | 6.03 ± 1.88 | 6.07 ± 1.67 | 0.683 |
| Daily | g | 230 ± 72 | 236 ± 63 | 0.164 |

^{xy}Different superscripts in same row are trending (x/y 0.05 < P ≤ 0.10).

Results show that the litter weight gain from cross-fostering up to weaning of sows feeding diets containing muramidase increased numerically by 4.5% in comparison to the control group. Regarding litter- or individually based daily weight gains in piglets from cross-fostering up to weaning, results in the muramidase group were about 2.6% greater than those noted in the control group. Results regarding the individual based dataset were nearly like those shown as litter-based means.

The mortality rate of piglets from cross-fostering up to weaning was in the range of 10% in the control and 7.7% in the muramidase group, respectively. The lower mortality rate in litters of sows fed diets containing muramidase resulted in greater amounts of piglets at weaning in comparison to the control (+3.4%; P: 0.066).

The reduced body weight loss in sows fed diets containing muramidase in combination with the increase in their litter weight gains despite of the lower feed intake compared to the control group might involve better utilization of nutrients, which was obviously reflected in a higher milk production. Most likely benefits on milk yield could be a hypothetical mechanism for slightly better growth and higher litter weaning weights. Since 4.1 kg milk is needed for 1 kg litter weight gain, hypothetical benefits of 3.16 kg litter weight gain in sows fed diets with muramidase addition are corresponding to approximately 12.96 kg enhanced milk production when compared to sows fed the lactation feed without using muramidase.

The cumulative intake of creep feed per litter amounted to an average of 1.37 kg or 72 g per day, without any differences among treatment groups. Since approximately 20.5 MJ ME is needed for 1 kg litter weight gain about 0.9 kg litter weight gain was supplied by the overall intake of creep feed which corresponded to about 1.27% of the overall litter weight gain. Therefore, the hypothetical benefit of muramidase on milk production seemed to be the most important way of slightly increased body weight gains of suckling piglets from cross-fostering up to weaning at d 26 of age.

### Conclusion

From the present study it can be concluded that the addition of muramidase to the lactation diet improved the litter size of piglets and reduced the mortality rate of piglets from cross-fostering up to weaning, resulting in a trend to greater amounts of piglets at weaning, and increased body weight gain of piglets in comparison to the control. Moreover, feed intake as well as body weight losses and back fat losses during the 26-d post-farrowing period were lower in sows fed diets containing muramidase in comparison to the control group. Estimations suggest that the application of muramidase increased milk production by approximately 12.96 kg per sow. Regarding overall calculated milk yield of about 285 kg per sow (control), the hypothetical benefit of muramidase on overall milk production amounted to approximately 4.55% in comparison to the control.

1. A method for improving health status and/or performance of a female animal during gestation and/or lactation and yong animals thereof, comprising administering to the female animal one or more microbial muramidases.

2. A method for reducing weight gain loss and/or backfat loss of a female animal during gestation and/or lactation, comprising administering to the female animal one or more microbial muramidases.

3. A method for increasing litter size of yong animals born by of a female animal and increasing milk yield of of a female animal, comprising administering to the female one or more microbial muramidases.

4. A method for improving weight gain and/or vadility of yong animals born by a female animal, comprising administering to the female animal one or more microbial muramidases.

5. The method of claim 1, wherein the microbial muramidase is dosed at a level of 100 to 1000 mg enzyme protein per kg animal feed, such as 200 to 900 mg, 300 to 800 mg, 400 to 700 mg, 500 to 600 mg enzyme protein per kg animal feed, or any combination of these intervals.

6. The method claim 1, wherein the animal is selected from the group consisting of swine such as sows and gilts; poultry such as turkey, duck, quail, guinea fowl, goose, pigeon, and chicken such as hens and pullets; cattle such as cows; cats; dogs; rabbits; horses; camels and sheep.

7. The method of claim 1, wherein the animal is sow or gilt.

8. The method of claim 1, wherein the microbial muramidase may be fed to the female animal during gestation and/or during lactation.

9. The method of claim 1, wherein the microbial muramidase is fed to sows or gilts from day 7 before farrowing to day 26 post-farrowing.

10. The method of claim 1, wherein the microbial muramidase is obtained or obtainable from the phylum Ascomycota, or the subphylum Pezizomycotina.

11. The method of claim 1, wherein the microbial muramidase comprises one or more domains selected from the list consisting of GH24 and GH25.

12. The method of claim 1, wherein the microbial muramidase is selected from the group consisting of:
(a) a polypeptide having at least 50%, e.g., at least 60%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 1;
(b) a variant of SEQ ID NO: 1 wherein the variant has muramidase activity and comprises one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions;
(c) a fragment of the polypeptide of (a) or (b) that has muramidase activity wherein the fragment comprises at least 170 amino acids, such as at least 175 amino acids, at least 177 amino acids, at least 180 amino acids, at least 185 amino acids, at least 190 amino acids, at least 195 amino acids or at least 200 amino acids;
(d) a polypeptide having at least 50%, e.g., at least 60%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 4;
(e) a variant of SEQ ID NO: 4 wherein the variant has muramidase activity and comprises one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions; and
(f) a fragment of the polypeptide of (d) or (e) that has muramidase activity wherein the fragment comprises at least 210 amino acids, such as at least 215 amino acids, at least 220 amino acids, at least 225 amino acids, at least 230 amino acids, at least 235 amino acids or at least 240 amino acids.

13. The method of claim 1, wherein the microbial muramidase is selected from the group consisting of amino acids 1 to 213 of SEQ ID NO: 1, amino acids 1 to 245 of SEQ ID NO: 4 and amino acids 1 to 208 of SEQ ID NO: 10.

14. A method for reducing weight gain loss and/or backfat loss of sows or gilts during gestation and/or lactation, comprising administering to the sows or gilts one or more microbial muramidases, wherein:
(a) the microbial muramidase is a GH24 or GH25 muramidase obtained or obtainable from the phylum Ascomycota, and is dosed at a level of 300 to 500 mg enzyme protein per kg animal feed; and
(b) the microbial muramidase is fed on a daily basis during gestation and lactation of the sows or gilts; and
(c) optionally the weight gain loss and/or backfat loss of the sows or gilts is reduced by at least 1%.

15. A method for increasing litter size of piglets born by sows or gilts, and/or increasing milk yield of sows or gilts, comprising administering to the sows or gilts one or more microbial muramidases, wherein:
(a) the microbial muramidase is a GH24 or GH25 muramidase obtained or obtainable from the phylum Ascomycota, and is dosed at a level of 300 to 500 mg enzyme protein per kg animal feed; and
(b) the microbial muramidase is fed on a daily basis during gestation and lactation of the sows or gilts; and
(c) optionally, the litter size of the piglets is increased by at least 3%, or the increasing milk yield of sows or gilts is increased by at least 4%.

16. A method for improving weight gain and/or vadility of piglets, comprising administering to sows or gilts one or more microbial muramidases, wherein:
(a) the microbial muramidase is a GH24 or GH25 muramidase obtained or obtainable from the phylum Ascomycota, and is dosed at a level of 300 to 500 mg enzyme protein per kg animal feed; and
(b) the microbial muramidase is fed on a daily basis during lactation of the sows or gilts; and
(c) optionally, weight gain and/or vadility of piglets is increased by at least 2%.

17. A feed composition comprising one or more microbial muramidases for improving health status and/or performance of a female animal during gestation and/or lactation and yong animals thereof.

18. Use of one or more muramidase in a feed composition, a feed additive or an animal feed for reducing weight gain loss and/or backfat loss of a female animal during gestation and/or lactation.

19. Use of claim 18, wherein the animal is selected from the group consisting of swine such as sows and gilts; poultry such as turkey, duck, quail, guinea fowl, goose, pigeon, and chicken such as hens and pullets; cattle such as cows; cats; dogs; rabbits; horses; camels and sheep. Preferably, the animal is a selected from the group consisting of swine, chicken, cats, dogs and sheep.

20. Use of claim 18, wherein the microbial muramidase is obtained or obtainable from the phylum Ascomycota, such as the sub-phylum Pezizomycotina.
